(19) United States
(12) Patent Application Publication
(10) Pub. No.: US 2022/0241481 A1
(43) Pub. Date: Aug. 4, 2022

(54) APPARATUS FOR PROVIDING DIALYSIS CONCENTRATE

(71) Applicant: Fresenius Medical Care Deutschland GmbH, Bad Homburg (DE)

(72) Inventor: Florian ENGELN, Laubach (DE)

(73) Assignee: Fresenius Medical Care Deutschland GmbH, Bad Homburg (DE)

(21) Appl. No.: 17/629,438

(22) PCT Filed: Jul. 23, 2020
(86) PCT No.: PCT/EP2020/070832

§ 371 Date: Jan. 24, 2022

(30) Foreign Application Priority Data
Jul. 25, 2019 (DE) 10 2019 120 174.8

(51) Int. Cl. A61M 1/16

(52) U.S. Cl. CPC A61M 1/1668; A61M 1/1666; A61M 2205/3334; A61M 2205/3317; A61M 2205/3324; A61M 2205/3306

(57) ABSTRACT

The present invention relates to an apparatus for providing liquid dialysis concentrate comprising a dialysis concentrate container for receiving the dialysis concentrate, wherein the dialysis concentrate container has an outflow and wherein the apparatus has a measurement device that is in fluid communication with the outflow and that is configured to determine the density or a property of the dialysis concentrate correlated with the density, with a shut-off element, in particular a valve, being arranged in the outflow by means of which the outflow can be selectively opened or closed.

The present invention relates to an apparatus for providing liquid dialysis concentrate comprising a dialysis concentrate container for receiving the dialysis concentrate.

It is known from the prior art to store liquid dialysis concentrate in one or more dialysis concentrate containers to prepare the ready-to-use dialysis solution from the dialysis concentrates by a corresponding dilution, typically with RO water.

Dry concentrates or suspended concentrates are typically presented in a swap container to prepare the dialysis concentrates. After the connection of the swap container to the apparatus, water is pumped into the swap container and the mixture is recirculated through a mixing tank and the swap container in a recirculation circuit until all the solids of the dry concentrate have been dissolved. Once this is the case, the liquid dialysis concentrate prepared in this manner is conveyed into one or more dialysis concentrate containers and is supplied from there to, for example, a central supply unit for dialysis concentrates.

An essential aspect in this procedure is the complete dissolving of the dry concentrate to ensure the desired composition of the liquid dialysis concentrate. Two variants are used to be able to check this: on the one hand, the removal of a sample of the dialysis concentrate and its sending to a laboratory and, on the other hand, the density measurement by the operator himself by means of a hand-held densitometer.

Both variants are laborious so that it is the underlying object of the present invention to further develop an apparatus of the initially named kind such that the density measurement is possible for the operator, i.e. for the user of the apparatus, in a particularly simple and time-saving manner.

It is furthermore known from the prior art, for example from the document EP 2 745 862 A1, to equip a mixing system for producing ready-to-use solutions of concentrates and high-purity water for use as a concentrate or a flushing solution for hemodialysis with an integrated measuring device to measure the density of the produced solutions.

The mixing system known from EP 2 745 862 A1 has a tank into which solvent, e.g. water, is metered. The solvent is subsequently conveyed into a tank with dry concentrate, whereupon the dry concentrate is brought into solution by a circulation of the suspension through the tank, the raw material container, and the lines connected thereto that together form a circulation circuit.

The measuring device for the density measurement is here arranged in the circulation circuit; a measurement chamber can be provided for this purpose, for example. The measuring device for the density measurement is thus an element of the mixing system, in particular of its circulation circuit, and is flowed around by the mixture of dry concentrate and solvent.

Such mixing systems are, however, very expensive and most preparation systems do not have any such integrated density measurement so that the density of the produced solutions typically has to be measured by hand in practice.

A further object of the present invention thus comprises providing a possibility of retrofitting existing preparation systems for a simple and time-saving density measurement by the user.

Worded in a general manner, the underlying object of the present invention thus comprises alleviating or even fully eliminating the problems of the prior art.

This object is achieved by an apparatus having the features of claim 1.

Provision is accordingly made that the dialysis concentrate container has an outflow and that the apparatus has a measurement device that is in fluid communication with the outflow and that is configured to determine the density or a property of the dialysis concentrate correlated with the density, with a shut-off element, in particular a valve, being arranged in the outflow by means of which the outflow can be selectively opened or closed.

In this respect, the dialysis concentration container is preferably not a part of the circulation circuit and is not used in the dissolving of a concentrate in a solvent, e.g. water. In other words, the dialysis concentrate container is preferably not flowed through by the mixture of dry concentrate and the solvent on the mixing of a concentrate and a solvent.

In other words, the dialysis concentrate container is preferably thus formed separately and/or independently of a circulation concentrate of a preparation system or mixing system and is not flowed through by a circulating mixture of concentrate and solvent.

The circulation circuit can, for example, have a mixing container and a swap container that are connected to one another via a (re)circulation line. The fully mixed dialysis concentrate is preferably conducted into the dialysis concentrate container after the recirculation.

The outflow can comprise a line, in particular a tube or a pipe, by means of which the dialysis concentrate is conducted from the dialysis concentrate container to the measurement device. If a density measurement or the measurement of a property is desired by means of which a conclusion can be drawn on the density (such as the light transmission, etc.), the shut-off element is opened so that the dialysis concentrate is conducted from the dialysis concentrate container to the measurement device.

The outflow is thus preferably not adapted to be connected to a preparation system and/or to a circulation circuit of a preparation system, but rather preferably only serves the emptying of the dialysis concentrate container. The present invention can thus be applied to every dialysis concentrate container.

This opening of the shut-off element can take place manually by the user as required or also in an automated manner by a control unit that instigates the opening of the shut-off element.

When a density measurement is spoken of in the following, this not only includes the measurement of the density of the dialysis concentrate, but rather also includes the measurement of every desired property from which a conclusion can be drawn on the density of the dialysis concentrate.

The light absorption of the dialysis concentrate, the transmission of light through the dialysis concentrate, the concentration of one, more, or all the ingredients of the dialysis concentrate, the conductivity of the dialysis concentrate, the presence of undissolved solids in the dialysis concentrate, or the viscosity of the dialysis concentrate, or a combination of the aforesaid parameters can be named by way of example as a property correlated with the density.

A backflow prevention element such as a check valve can be arranged in the outflow.

Provision is made in a preferred embodiment of the invention that a plurality of dialysis concentrate containers are provided that each have an outflow, with the plurality of outflows being brought together in a manifold that is arranged upstream of the measurement device in the direction of flow of the dialysis concentrate.

Every dialysis concentrate container is in this case preferably equipped with a shut-off element such as with a sampling faucet. Concentrate tubes that are brought together in a manifold block or the like, but are fluidically separate from one another, are connected to these shut-off elements.

One shut-off element or also a plurality of shut-off elements can be provided per outflow.

The shut-off element or elements can be arranged at or in the manifold. It is thus conceivable, for example, to arrange a respective sampling faucet or the like at the dialysis concentrate container or containers and to arrange a respective spherical valve or the like, and optionally a backflow prevention mechanism, in front of the inlet into the manifold.

Provision is preferably further made that a concentrate tube or another concentrate line is connected, leading away from the manifold, to an inline densitometer, i.e. to the measurement device in accordance with the invention.

It is conceivable that the corresponding spherical valve is opened at the manifold with an open sampling faucet in order to determine the density of the dialysis concentrate contained in the dialysis concentrate container so that the concentrate flows out of the respective dialysis concentrate container through the densitometer and from there preferably into the drain.

It is preferred if the outflow projects into a fluid, e.g. into a siphon, without a gravity path in its end region so that the system is free of air. The total system is thus permanently free of air and possible internal crystallizations and blockages of the concentrate can thus be avoided.

Provision is made in a further embodiment of the invention that the apparatus has a reading device or an input device by means of which the concentrate type from which the dialysis concentrate is prepared can be read in or input and that a database or another store is present in which a correlation between the concentrate type and the density or the property correlated therewith of the dialysis concentrate is stored.

The concentrate type of the concentrate, preferably of the dry concentrate, that is present in a corresponding concentrate container, that is also called a swap container in the following, is determined by means of the reading device. The reading device is, for example, a hand-held barcode reader or another scanner.

It is connected to a PC or the like, as is the measurement device, preferably a densitometer, and optionally a label printer and/or a signaling device by means of which it is possible to signal to the user whether the dialysis concentrate has the correct density and thus a complete dissolution of the dry concentrate has taken place.

A comparison unit can be provided for this purpose that compares the measured density/property with the density/property stored in the database.

The comparison unit can be configured to output a signal dependent on the comparison and perceptible to the user and/or to print a label on which the data relating to the dialysis concentrate are printed. The latter preferably only takes place when the measured density or the property correlated therewith is within a desired value range.

To ensure that the measurement device measures the density or the like of the correct dialysis concentrate and not residues of a preceding measurement, it is preferred if a control is present that only activates the measurement device after expiration of a certain time period after the opening of the shut-off element or after another action. It is thus conceivable, for example, that the control only carries out the density measurement 30 seconds or similar after the opening of the spherical valve or after another trigger event.

It is conceivable that the control is configured such that the measurement device is only activated after a specific time period after the activation of the reading device and/or of the input device and carries out the measurement of the density or of the other property of the liquid dialysis concentrate.

The apparatus preferably has a mixing container and a swap container in which the concentrate from which the dialysis concentrate is prepared is contained. The swap container serves the reception of the concentrate, that is preferably present as a dry concentrate, from which the liquid dialysis concentrate is prepared.

It is pointed out at this point that the invention covers both the case that the containers, tanks, etc. are filled and the case that this is not the case. The apparatus per se and the apparatus in the filled state are thus covered by the present invention.

In the filled state, the concentrate (preferably dry concentrate) from which the liquid dialysis concentrate is prepared is in the swap container and the mixing or dissolving of this concentrate with water takes place in the recirculation circuit that includes the mixing container, a pump, and the swap container. The liquid dialysis concentrate from which the ready-to-use dialysis solution is then prepared in a further step is present in the dialysis concentrate container.

The mixing container and the swap container are preferably in fluid communication with one another by a recirculation line.

Provision is furthermore preferably made that the mixing container is in fluid communication with the dialysis concentrate container or containers to transfer the dialysis concentrate from the mixing container into the dialysis concentrate container or containers by means of a pump.

Provision can furthermore be made that a plurality of dialysis concentrate containers are present in which dialysis concentrates each having different compositions are contained.

It is pointed out at this point that the terms “a” and “one” do not necessarily refer to exactly one of the elements, even though this represents a possible embodiment, but can also designate a plurality of elements. The use of the plural equally also includes the presence of the element in question in the singular and, conversely, the singular also includes a plurality of the elements in question.

Further details and advantages of the invention will be explained in more detail with reference to an embodiment shown in the drawing.

There are shown:

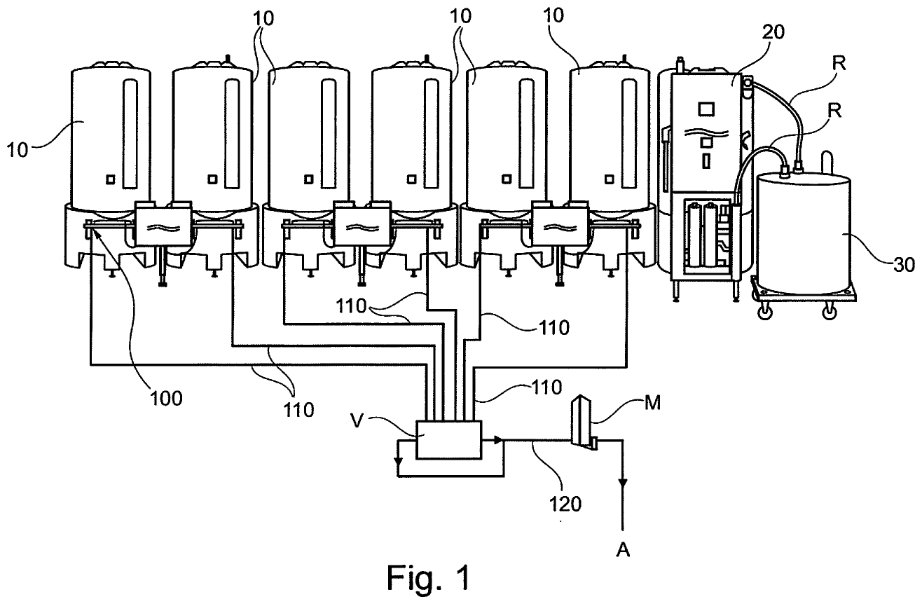

FIG. 1: a schematic view of the apparatus in accordance with the invention with a plurality of dialysis concentrate containers;

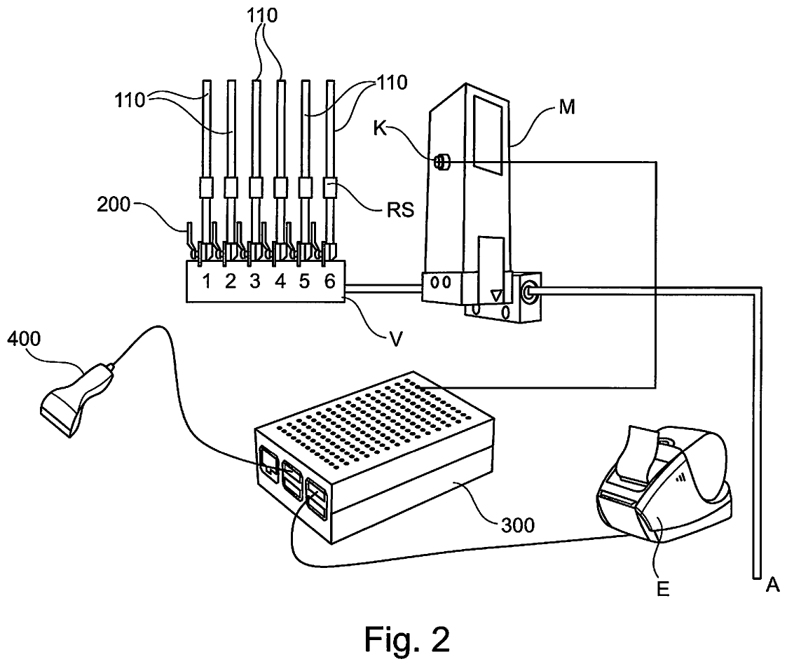

FIG. 2: a schematic representation of the manifold block with a measurement device, PC, scanner, and label printer;

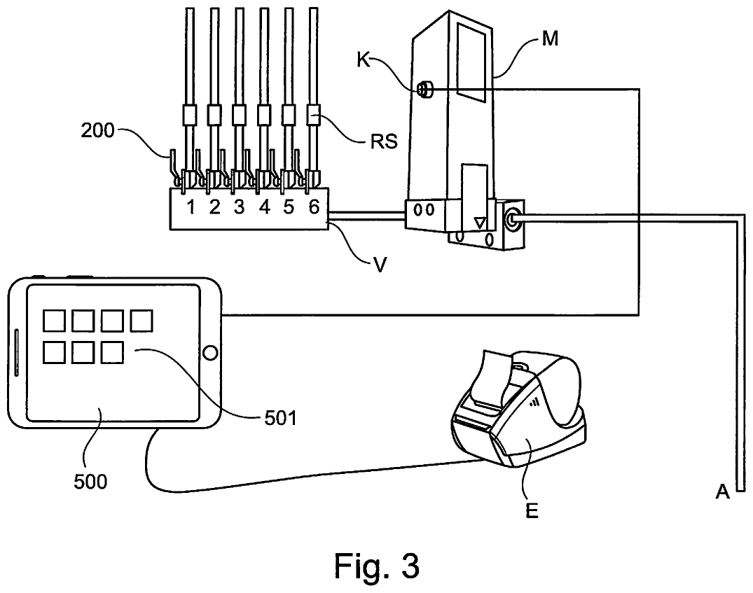

FIG. 3: a schematic representation of the manifold block with a measurement device, tablet, and label printer; and

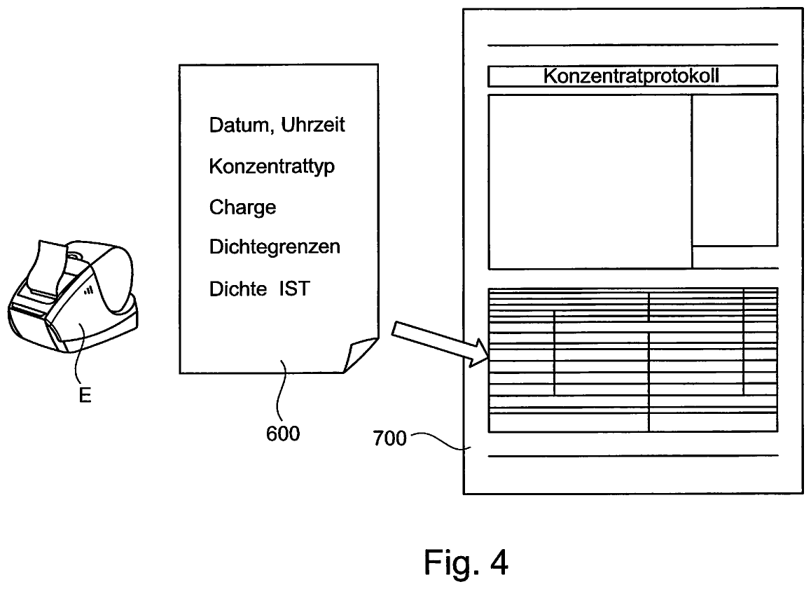

FIG. 4: an exemplary representation of the label printer and of a label for the concentrate report.

FIG. 1 shows an apparatus in accordance with the invention having a total of six dialysis concentrate containers 10 in which the liquid dialysis concentrate is present. A mixing container 20 is furthermore provided that is connected to a swap container 30 via recirculation lines R.

A dry concentrate or a suspended concentrate is present in the swap container 30 that is mixed with water, preferably RO water, from the mixing container 20 and is dissolved by recirculation between the swap container 30 and the mixing container 20. The recirculation is effected by a pump not shown in any more detail.

After the dissolving of the dry concentrate or of the suspended concentrate, the dialysis concentrate moves into the dry concentrate container 10 by means of a pump, not shown in any more detail, with the same dialysis concentrate or also different dialysis concentrates being able to be present in all the dialysis concentrate containers.

Every dialysis concentrate container 10 is equipped with a sampling faucet 100, with it only being provided with a reference numeral at the dialysis concentrate container 10 shown at the left for better clarity.

The sampling faucets 100 are used for the purpose of removing concentrate samples. Concentrate tubes 110 are connected to all the sampling faucets 100 (six units here). They are brought together at a manifold block V, as can be seen from FIG. 1.

A respective spherical valve 200 and a respective backflow prevention mechanism RS are located in front of the entrance into the manifold block V, as can be seen from FIGS. 2 and 3. It can also be seen from these Figures that every tube 110 is connected to the manifold V via its own connector.

Leading away from the manifold block V, a concentrate tube 120 is connected to an inline densitometer M, as is shown in FIGS. 1 to 3.

The corresponding spherical valve 200 is opened at the manifold block V to determine the density of the liquid dialysis concentrate present in the dialysis concentrate container 10. The dialysis concentrate thus flows through the densitometer M and from there into the drain A.

The end of the drain tube 120 projects into a liquid, e.g. into a siphon, without a gravity path. The total concentrate sampling system is thus permanently free of air. Possible internal crystallizations and blockages of the concentrate can thus be avoided.

FIG. 2 shows a PC, preferably a mini-PC, by reference numeral 300 having special software in which all the concentrate types (e.g. seven) are stored with the associated density thresholds and temperature correction values. The following hardware is connected to the PC 300: the densitometer M, a hand-held barcode reader 400, and a (mini) label printer E, all via an RD 232 connector K.

After the opening of the spherical valve 200, the dialysis concentrate flows from the dialysis concentrate container 100 to be sampled, for example at 200 ml/min, through the densitometer M into the drain A.

Once the user has scanned the label at the swap container 30 by the barcode reader 400, the software 30 counts 30 seconds (or another suitable time period) until the transfer, i.e. measurement of the current density value and temperature value. It is ensured after these 30 seconds that all the filling volumes remaining from the last measurement have left the system.

If the measured density value is within the tolerance limits, a corresponding label printout takes place such as is shown in FIG. 4. The date, the time of the measurement, the concentrate type, the batch, the density limits, and the measured value of the density are indicated on the label 600. Reference numeral 700 in FIG. 4 shows a concentrate record.

A tablet can also be used instead of a reading device, as is shown in FIG. 3. An app having a selection of the concentrate type is present on the tablet 500 and all the concentrate types (e.g. seven) are stored in it together with the associated density thresholds and temperature correction values. The following hardware is connected to the tablet 500: The densitometer M and the (mini) label printer E.

After the opening of the spherical valve 200, the dialysis concentrate flows from the dialysis concentrate container 10 to be sampled, e.g. at 200 ml/min, through the densitometer M into the drain A, as also in the procedure described with reference to FIG. 2.

Once the user has selected the concentrate type used from the selection 501 on the display of the tablet 500, the software counts 30 seconds up to the transfer, i.e. the measurement of the current density value and temperature value.

It is ensured after these 30 seconds that all the filling volumes remaining from the last measurement have left the system. If the density value is within the tolerance limits, a corresponding label printout takes place such as was already described with reference to FIG. 4.

The measurement arrangement in accordance with the invention can be arranged at the apparatus from the start or can be installed as a retrofit kit at existing apparatus to prepare a liquid dialysis concentrate. The dialysis concentrate containers must be provided with an outflow for this purpose and the described measurement equipment must be provided.

1. An apparatus for providing liquid dialysis concentrate comprising a dialysis concentrate container for receiving the dialysis concentrate, characterized in that the dialysis concentrate container has an outflow; and in that the apparatus has a measurement device that is in fluid communication with the outflow and that is configured to determine the density or a property of the dialysis concentrate correlated with the density, with a shut-off element being arranged in the outflow by means of which the outflow can be selectively opened or closed.

2. An apparatus in accordance with claim 1, characterized in that the measurement device is configured to measure as the property correlated with the density the light absorption of the dialysis concentrate, the transmission of light through the dialysis concentrate, the concentration of one, more, or all the ingredients of the dialysis concentrate, the conductivity of the dialysis concentrate, the presence of undissolved solids in the dialysis concentrate, or the viscosity of the dialysis concentrate, or a combination of the aforesaid parameters.

3. An apparatus in accordance with claim 1, characterized in that a back flow prevention element is arranged in the outflow; and/or in that a sampling faucet is arranged at the dialysis concentrate container and/or in the outflow downstream of the dialysis concentrate as a shut-off element container.

4. An apparatus in accordance with claim 1, characterized in that a plurality of dialysis concentrate containers are provided that each have an outflow, with the plurality of outflows being brought together in a manifold that is arranged upstream of the measurement device in the direction of flow of the dialysis concentrate.

5. An apparatus in accordance with claim 4, characterized in that one or more of the shut-off elements are arranged at or in the manifold.

6. An apparatus in accordance with claim 1, characterized in that the outflow projects into a liquid without a gravity path in its end region so that the system is free of air.

7. An apparatus in accordance with claim 1, characterized in that the apparatus has a reading device or an input device by means of which the concentrate type from which the dialysis concentrate is prepared can be read in or input; and in that a store is present in which a correlation between the concentrate type and the density or the property correlated therewith of the dialysis concentrate is stored.

8. An apparatus in accordance with claim 7, characterized in that a comparison unit is present that is configured to compare the measured density or the property correlated therewith with the density or property stored in the store.

9. An apparatus in accordance with claim 8, characterized in that the comparison unit is configured to output a signal dependent on the comparison and perceptible to the user and/or to initiate the printing of a label on which the data relating to the dialysis concentrate are printed.

10. An apparatus in accordance with claim 1, characterized in that a control is present that only activates the measurement device expiration of a specific time period after the opening of the shut-off element.

11. An apparatus in accordance with claim 1, characterized in that a control is present that only activates the measurement device after activation or actuation of the reading device and/or of the input device.

12. An apparatus in accordance with claim 1, characterized in that the apparatus has a mixing container and a swap container in which the concentrate, preferably the dry concentrate, from which the dialysis concentrate is prepared is contained or receivable.

13. An apparatus in accordance with claim 12, characterized in that the mixing container and the swap container are in fluid communication with one another by a recirculation line; and in that the mixing container is in fluid communication with the dialysis concentrate container or containers to transfer the dialysis concentrate from the mixing container into the dialysis concentrate container or containers.

14. An apparatus in accordance with claim 1, characterized in that a plurality of dialysis concentrate containers are present in which dialysis concentrates each having different compositions are contained.
